SEQUENCE LISTING (33 sequences)

SEQ ID NO: 1 - DNA, 7056 bp, Homo sapiens
cacacccacggcagacacgcacgcacccgggcgccgaagggaaagccgcgtctcgccctcccgccccgccgtcggtcctgtctcagtccctcagcagagcgggaaagcggaggccggagccgtgacctctgaccccgtggttatgcggagccgccgcattccttagcgatcgcggggcagccgccgctgccgccgtgggcgactgacgcagcgcgggcgcgtggagccgccgccgcccctcccccaccgccgctctcgcgccagccggtccccgcgtgcccgccccttctccccggccgcacccgagacctcgcgcgccgccgctgccacgcgccccccccaccgccgccgccgccccagccccgcgccaccgccccagcccgcccagcccggaggtcccgcgtggagctgccgccgccgccggggagaaggatgaaggacaaacagaagaagaagaaggagcgcacgtgggccgaggccgcgcgcctggtattagaaaactactcggatgctccaatgacaccaaaacagattctgcaggtcatagaggcagaaggactaaaggaaatgagaagtgggacttcccctctcgcatgcctcaatgctatgctacattccaattcaagaggaggagaggggttgttttataaactgcctggccgaatcagccttttcacgctcaagaaggatgccctgcagtggtctcgccatccagctacagtggagggagaggagccagaggacacggctgatgtggagagctgtgggtctaatgaagccagcactgtgagtggtgaaaacgatgtatctcttgatgaaacatcttcgaacgcatcctgttctacagaatctcagagtcgacctctttccaatcccagggacagctacagagcttcctcacaggcgaacaaacaaaagaaaaagactggggtgatgctgcctcgagttgtcctgactcctctgaaggtaaacggggcccacgtggaatctgcatcagggttctcgggctgccacgccgatggcgagagcggcagcccgtccagcagcagcagcggctctctggccctgggcagcgctgctattcgtggccaggccgaggtcacccaggaccctgccccgctcctgagaggcttccggaagccagccacaggtcaaatgaagcgcaacagaggggaagaaatagattttgagacacctgggtccattcttgtcaacaccaacctccgtgccctgatcaactctcggaccttccatgccttaccatcacacttccagcagcagctcctcttcctcctgcctgaagtagacagacaggtggggacggatggcctgttgcgtctcagcagcagtgcactaaataacgagttttttacccatgcggctcagagctggcgggagcgcctggctgatggtgaatttactcatgagatgcaagtcaggatacgacaggaaatggagaaggaaaagaaggtggaacaatggaaagaaaagttctttgaagactactatggacagaagctgggtttgaccaaagaagagtcattgcagcagaacgtgggccaggaggaggctgaaatcaaaagtggcttgtgtgtcccaggagaatcagtgcgtatacagcgtggtccagccacccgacagcgagatgggcattttaagaaacgctctcggccagatctccgaaccagagccagaaggaatctgtacaaaaaacaggagtcagaacaagcaggggttgctaaggatgcaaaatctgtggcctcagatgttcccctctacaaggatggggaggctaagactgacccagcagggctgagcagtccccatctgccaggcacatcctctgcagcacccgacctggagggtcccgaattcccagttgagtctgtggcttctcggatccaggctgagccagacaacttggcacgtgcctctgcatctccagacagaattcctagcctgcctcaggaaactgtggatcaggaacccaaggatcagaagaggaaatcctttgagcaggcggcctctgcatcctttcccgaaaagaagccccggcttgaagatcgtcagtcctttcgtaacacaattgaaagtgttcacaccgaaaagccacagcccactaaagaggagcccaaagtcccgcccatccggattcaactttcacgtatcaaaccaccctgggtggttaaaggtcagcccacttaccagatatgcccccggatcatccccaccacggagtcctcctgccggggttggactggcgccaggaccctcgcagacattaaagcccgtgctctgcaggtccgaggggcgagaggtcaccactgccatagagaggcggccaccactgccatcggaggggggggtggcccgggtggaggtggcggcggggccaccgatgagggaggtggcagaggcagcagcagtggtgatggtggtgaggcctgtggccaccctgagcccaggggaggcccgagcacccctggaaagtgtacgtcagatctacagcgaacacaactactgccgccttatcctctaaatggggagcatacccaggccggaactgccatgtccagagctaggagagaggacctgccttctctgagaaaggaggaaagctgcctactacagagggctacagttggactcacagatgggctaggagatgcctcccaactccccgttgctcccactggggaccagccatgccaggccttgcccctactgtcctcccaaacctcagtagctgagagattagtggagcagcctcagttgcatccggatgttagaactgaatgtgagtctggcaccacttcctgggaaagtgatgatgaggagcaaggacccaccgttcctgcagacaatggtcccattccgtctctagtgggagatgatacattagagaaaggaactggccaagctcttgacagtcatcccactatgaaggatcctgtaaatgtgacccccagttccacacctgaatcctcaccgactgattgcctgcagaacagagcatttgatgacgaattagggcttggtggctcatgccctcctatgagggaaagtgatactagacaagaaaacttgaaaaccaaggctctcgtttctaacagttctttgcattggatacccatcccatcgaatgatgaggtagtgaaacagcccaaaccagaatccagagaacacataccatctgttgagccccaggttggagaggagtgggagaaagctgctcccacccctcctgcattgcctggggatttgacagctgaggagggtctagatcctcttgacagccttacttcactctggactgtgccatctcgaggaggcagtgacagcaatggcagttactgtcaacaggtggacattgaaaagctgaaaatcaacggagactctgaagcactgagtcctcacggtgagtccacggatacagcctctgactttgaaggtcacctcacggaggacagcagtgaggctgacactagagaagctgcagtgacaaagggatcttcggtggacaaggatgagaaacccaattggaaccaatctgccccactgtccaaggtgaatggtgacatgcgtctggttacaaggacagatgggatggttgctcctcagagctgggtgtctcgagtatgtgcggtccgccaaaagatcccagattccctactgctggccagtactgagtaccagccaagagccgtgtgcctgtccatgcctgggtcctcagtggaggccactaacccacttgtgatgcagttgctgcagggtagcttgcccctagagaaggttcttccaccagcccacgatgacagcatgtcagaatccccacaagtaccacttacaaaagaccagagccatggctcgctacgcatgggatctttacatggtcttggaaaaaacagtggcatggttgatggaagcagccccagttctttaagggctttgaaggagcctcttctgccagatagctgtgaaacaggcactggtcttgccaggattgaggccacccaggctcctggagcaccccaaaagaattgcaaggcagtcccaagttttgactccctccatccagtgacaaatcccattacatcctctaggaaactggaagaaatggattccaaagagcagttctcttcctttagttgtgaagatcagaaggaagtccgtgctatgtcacaggacagtaattcaaatgctgctccaggaaagagcccaggagatcttactacctcgagaacacctcgtttctcatctccaaatgtgatctcctttggtccagagcagacaggtcgggccctgggtgatcagagcaatgttacaggccaagggaagaagctttttggctctgggaatgtggctgcaacccttcagcgccccaggcctgcggacccgatgcctcttcctgctgagatccctccagtttttcccagtgggaagttgggaccaagcacaaactccatgtctggtggggtacagactccaagggaagactgggctccaaagccacatgcctttgttggcagcgtcaagaatgagaagacttttgtggggggtcctcttaaggcaaatgccgagaacaggaaagctactgggcatagtcccctggaactggtgggtcacttggaagggatgccctttgtcatggacttgcccttctggaaattaccccgagagccagggaaggggctcagtgagcctctggagccttcttctctcccctcccaactcagcatcaagcaggcattttatgggaagctttctaaactccaactgagttccaccagctttaattattcctctagctctcccacctttcccaaaggccttgctggaagtgtggtgcagctgagccacaaagcaaactttggtgcgagccacagtgcatcactttccttgcaaatgttcactgacagcagcacggtggaaagcatctcgctccagtgtgcgtgcagcctgaaagccatgatcatgtgccaaggctgcggtgcgttctgtcacgatgactgtattggaccctcaaagctctgtgtattgtgccttgtggtgagataataaattatggccatgggaaacattgtatatttagtgtgtgtattttgataatgattgatcttaaatctgtatacagaatatcattgatataatactctttaggcaggagcactcttgccttcccccaaaatttacactgctaaagccctctgtcacttggcgacccttctggtcttgctggaggggtttcctgggtataacccattgggctgcccaaggccagccagcctgagctctcctgcaagacagagcctgatgtggcacggagtggggttgcggggggtggggggactgcctgactcccagagggacttgaaactgaagcaagaaggttgcattctccaccaagggagttaacctacctgaactaagtagaaatgccagtcttccactaccccctccctgccatcttttcttctgctactttggggagttgatggccaggaaagaagccagcacagggttaaagtaactcctggcattgcccaccagggggctggtgcacctgctgacctcagggtcacagttgagtcatttgccagttgacggagcaagtttgaccttggttctgttgctgaagcaaatttggaacttttctgtctcagtgtgatccactaacccacaggatcatttggaaccttgaatagctctgcttggacaatggggttggggaatagggttgtctttcctatgaaaatgccatctgtagaccttgtgagtcagccgtccagatgtttgcaggtgaattcctctgcttgacatcctccctgtcactttggaccctatgggagtgggcatctccacgcacctgtgtatgtgaaagtcattttacatttcaaagcagtgtgtgtttcttatttttatatttttaactctttattcttggatgtataaagtgaactttttggcttctgtaagtatgctctatgcacctctaatgttttatcatgtatttatatgttgtacacagtactggctgattctgtaaatggatgtattgtacagagaacatgaacgtctcttcctaattttacatcttcagcatcattgcattaaagtggtgtaatctccttctctacatctgttgtcagagccactgagtgctgtgctgctcgacgtgagggtgaaatgattgacttgtgacctgccaggttgcccgatgccctgttgggtcaccggctggacctgctgcagcctgcagagccacagtcagcctgcccacatgccaccgagcaaacgcatcttgcttttcacatctctcctcctacagccttaatggctgcttgctgccatatgtgacaaatcaccaccaccagtgttaagtgcttctggattcatgggtgagttccctgggcagcccccaggaaggccttccagatctggctccagggtcaccacctgtcacagcaatacctgggaccatgctctcctgggactgtgaggctccttttgacgtacttttgacatcaggcaggtttgggaagaaacaaagccatgcctgctcctgcctctctcccaacatgtttccagcaagtagatgcccctgtgtgtgttttcccttgccttgtttcctgccttatatcttgtatttcgacttattacagagttgagggttcttgcttaatttagatcaagtataaaatttgtatgacttcaagtctcattttatctgaaaggtttttttctcatttaatctgatgtggcattttcgtcatctgaagcatgagtgacaagttgggaatgatgtggtgatttagaatgcagtattggccaagtccaagttgtcaacttaagcgtctgtttaccaaagaccgggaacaggggcccaaacatgtccagtcctcttcttccctctgctggaacctttggggacactcaagggtacagtttgacactgatctggtccatgaggctgcccagagaaagcactgcttctgtatgtctcttgtggtattggaacaataaacccgtacaacctgcaaaaaaaaaaaaaaaaaa

SEQ ID NO: 2 - protein, 1541 aa, Homo sapiens
MKDKQKKKKERTWAEAARLVLENYSDAPMTPKQILQVIEAEGLKEMRSGTSPLACLNAMLHSNSRGGEGLFYKLPGRISLFTLKKDALQWSRHPATVEGEEPEDTADVESCGSNEASTVSGENDVSLDETSSNASCSTESQSRPLSNPRDSYRASSQANKQKKKTGVMLPRVVLTPLKVNGAHVESASGFSGCHADGESGSPSSSSSGSLALGSAAIRGQAEVTQDPAPLLRGFRKPATGQMKRNRGEEIDFETPGSILVNTNLRALINSRTFHALPSHFQQQLLFLLPEVDRQVGTDGLLRLSSSALNNEFFTHAAQSWRERLADGEFTHEMQVRIRQEMEKEKKVEQWKEKFFEDYYGQKLGLTKEESLQQNVGQEEAEIKSGLCVPGESVRIQRGPATRQRDGHFKKRSRPDLRTRARRNLYKKQESEQAGVAKDAKSVASDVPLYKDGEAKTDPAGLSSPHLPGTSSAAPDLEGPEFPVESVASRIQAEPDNLARASASPDRIPSLPQETVDQEPKDQKRKSFEQAASASFPEKKPRLEDRQSFRNTIESVHTEKPQPTKEEPKVPPIRIQLSRIKPPWVVKGQPTYQICPRIIPTTESSCRGWTGARTLADIKARALQVRGARGHHCHREAATTAIGGGGGPGGGGGGATDEGGGRGSSSGDGGEACGHPEPRGGPSTPGKCTSDLQRTQLLPPYPLNGEHTQAGTAMSRARREDLPSLRKEESCLLQRATVGLTDGLGDASQLPVAPTGDQPCQALPLLSSQTSVAERLVEQPQLHPDVRTECESGTTSWESDDEEQGPTVPADNGPIPSLVGDDTLEKGTGQALDSHPTMKDPVNVTPSSTPESSPTDCLQNRAFDDELGLGGSCPPMRESDTRQENLKTKALVSNSSLHWIPIPSNDEVVKQPKPESREHIPSVEPQVGEEWEKAAPTPPALPGDLTAEEGLDPLDSLTSLWTVPSRGGSDSNGSYCQQVDIEKLKINGDSEALSPHGESTDTASDFEGHLTEDSSEADTREAAVTKGSSVDKDEKPNWNQSAPLSKVNGDMRLVTRTDGMVAPQSWVSRVCAVRQKIPDSLLLASTEYQPRAVCLSMPGSSVEATNPLVMQLLQGSLPLEKVLPPAHDDSMSESPQVPLTKDQSHGSLRMGSLHGLGKNSGMVDGSSPSSLRALKEPLLPDSCETGTGLARIEATQAPGAPQKNCKAVPSFDSLHPVTNPITSSRKLEEMDSKEQFSSFSCEDQKEVRAMSQDSNSNAAPGKSPGDLTTSRTPRFSSPNVISFGPEQTGRALGDQSNVTGQGKKLFGSGNVAATLQRPRPADPMPLPAEIPPVFPSGKLGPSTNSMSGGVQTPREDWAPKPHAFVGSVKNEKTFVGGPLKANAENRKATGHSPLELVGHLEGMPFVMDLPFWKLPREPGKGLSEPLEPSSLPSQLSIKQAFYGKLSKLQLSSTSFNYSSSSPTFPKGLAGSVVQLSHKANFGASHSASLSLQMFTDSSTVESISLQCACSLKAMIMCQGCGAFCHDDCIGPSKLCVLCLVVR

SEQ ID NO: 3 - protein, 36 aa, Homo sapiens
CACSLKAMIMCQGCGAFCHDDCIGPSKLCVLCLVVR

SEQ ID NO: 4 - DNA, 22 bp, Artificial Sequence
  Primer ASXL1 ex1 F
tgacctctgaccccgtggttat

SEQ ID NO: 5 - DNA, 25 bp, Artificial Sequence
  Primer ASXL1 Ex1 R
ggctgtcccgttcttaaaggaagat

SEQ ID NO: 6 - DNA, 24 bp, Artificial Sequence
  Primer ASXL1 Ex2 F
tcaccagcggtacctcatagcata

SEQ ID NO: 7 - DNA, 24 bp, Artificial Sequence
  Primer ASXL1 Ex2 R
tctcactaccagacacagaagcac

SEQ ID NO: 8 - DNA, 25 bp, Artificial Sequence
  Primer ASXL1 Ex3 F
gaatgagttggagggattaggtctt

SEQ ID NO: 9 - DNA, 24 bp, Artificial Sequence
  Primer ASXL1 Ex3 R
ttcatggcccctattccagaccta

SEQ ID NO: 10 - DNA, 20 bp, Artificial Sequence
  Primer ASXL1 Ex4-5 F
cacctgagttgtaccttgct

SEQ ID NO: 11 - DNA, 24 bp, Artificial Sequence
  Primer ASXL1 Ex4-5 R
gggatcttgccttatacctgtcat

SEQ ID NO: 12 - DNA, 24 bp, Artificial Sequence
  Primer ASXL1 Ex6 F
ctgcagttgacttgggctctcttt

SEQ ID NO: 13 - DNA, 21 bp, Artificial Sequence
  Primer ASXL1 Ex6 R
tcatccattggcagggctctt

SEQ ID NO: 14 - DNA, 24 bp, Artificial Sequence
  Primer ASXL1 Ex7 F
gctgggagaaatgagcttgtctga

SEQ ID NO: 15 - DNA, 22 bp, Artificial Sequence
  Primer ASXL1 Ex7 R
aatagagggccacccaagaagt

SEQ ID NO: 16 - DNA, 24 bp, Artificial Sequence
  Primer ASXL1 Ex8-9 F
ggtgctgtcaccaaagttttccca

SEQ ID NO: 17 - DNA, 24 bp, Artificial Sequence
  Primer ASXL1 Ex8-9 R
aaccctttgagaagagcgagcttg

SEQ ID NO: 18 - DNA, 22 bp, Artificial Sequence
  Primer ASXL1 Ex10 F
actgggagattcagctgtccat

SEQ ID NO: 19 - DNA, 23 bp, Artificial Sequence
  Primer ASXL1 Ex10 R
tatcccctcaaaaccccagacca

SEQ ID NO: 20 - DNA, 24 bp, Artificial Sequence
  Primer ASXL1 Ex11 F
ggcctgaaactgatggctgtgatt

SEQ ID NO: 21 - DNA, 24 bp, Artificial Sequence
  Primer ASXL1 Ex11 R
tgaccctcaaagaaaacctggctc

SEQ ID NO: 22 - DNA, 22 bp, Artificial Sequence
  Primer ASXL1 Ex12-1 F
aggtcagatcacccagtcagtt

SEQ ID NO: 23 - DNA, 24 bp, Artificial Sequence
  Primer ASXL1 Ex12-1 R
tagcccatctgtgagtccaactgt

SEQ ID NO: 24 - DNA, 24 bp, Artificial Sequence
  Primer ASXL1 Ex12-2 F
agaggacctgccttctctgagaaa

SEQ ID NO: 25 - DNA, 24 bp, Artificial Sequence
  Primer ASXL1 Ex12-2 R
ttcgatgggatgggtatccaatgc

SEQ ID NO: 26 - DNA, 22 bp, Artificial Sequence
  Primer ASXL1 Ex12-3 F
acttgaaaaccaaggctctcgt

SEQ ID NO: 27 - DNA, 24 bp, Artificial Sequence
  Primer ASXL1 Ex12-3 R
gcaaccatcccatctgtccttgta

SEQ ID NO: 28 - DNA, 24 bp, Artificial Sequence
  Primer ASXL1 Ex12-4 F
ggtggacaaggatgagaaacccaa

SEQ ID NO: 29 - DNA, 24 bp, Artificial Sequence
  Primer ASXL1 Ex12-4 R
tgtcctgtgacatagcacggactt

SEQ ID NO: 30 - DNA, 25 bp, Artificial Sequence
  Primer ASXL1 Ex12-5 F
tggattccaaagagcagttctcttc

SEQ ID NO: 31 - DNA, 24 bp, Artificial Sequence
  Primer ASXL1 Ex12-5 R
catgacaaagggcatcccttccaa

SEQ ID NO: 32 - DNA, 24 bp, Artificial Sequence
  Primer ASXL1 Ex12-6 F
acaggaaagctactgggcatagtc

SEQ ID NO: 33 - DNA, 24 bp, Artificial Sequence
  Primer ASXL1 Ex12-6 R
caagagtgctcctgcctaaagagt

(19) United States
(12) Patent Application Publication
(10) Pub. No.: US 2015/0125868 A1
(43) Pub. Date: May 7, 2015

(54) ASXL1 AS A NEW DIAGNOSTIC MARKER OF MYELOID NEOPLASMS

(71) Applicants: Qiagen Marseille SA, Marseille Cedex 09 (FR); Institut Paoli-Calmettes, Marseille (FR); INSERM (Institut National de la Santé et de la Recherche Médicale), Paris Cedex 13 (FR)

(72) Inventors: VÉRONIQUE GELSI-BOYER, MARSEILLE (FR); DANIEL BIRNBAUM, LA BOUILLADISSE (FR)

(73) Assignees: Qiagen Marseille SA, Marseille Cedex 09 (FR); Institut Paoli-Calmettes, Marseille (FR); INSERM (Institut National de la Santé et de la Recherche Médicale), Paris Cedex 13 (FR)

(21) Appl. No.: 14/511,375

(22) Filed: Oct. 10, 2014

Related U.S. Application Data
(63) Continuation of application No. 13/578,667, filed on Nov. 19, 2012, now abandoned.
(60) Provisional application No. 61/303,971, filed on Feb. 12, 2010.

(51) Int. Cl. C12Q 1/68; C07K 16/32

(52) U.S. Cl. CPC C12Q 1/6886; C07K 16/32; C12Q 2600/156; C12Q 2600/106
USPC 435/6.12; 536/24.31; 536/24.33; 530/387.7

(57) ABSTRACT

The present invention relates to a method for diagnosing a myeloid cancer in a subject, which comprises the step of analyzing a biological sample from said subject by determining the presence or the absence of a mutation in the ASXL1 (additional sex combs like 1) gene coding for the polypeptide having the sequence SEQ ID No 2. A kit for diagnosing myeloid cancer in a subject comprising at least one nucleic acid probe or oligonucleotide or at least one antibody, which can be used in a such a method.

This application is a continuation of U.S. patent application Ser. No. 13/578,667, filed Nov. 19, 2012, which is a national stage filing under 35 U.S.C. §371 of international PCT application PCT/IB2011/000255, filed Feb. 11, 2011, which claims the benefit of U.S. Provisional Application No. 61/303,971 filed Feb. 12, 2010. The entire contents of each of the prior applications is incorporated by reference herein in its entirety.

### FIELD OF THE INVENTION

The present invention relates to genetic markers to diagnose myeloid neoplasms, more particularly to a new identified tumor suppressor gene. More particularly, the invention relates to ASXL1a new marker, which is useful for diagnosing MDS, CMML, MPN and AML.

### BACKGROUND

Hematopoiesis is maintained by a hierarchical system where hematopoietic stem cells (HSCS) give rise to multipotent progenitors, which in turn differentiate into all types of mature blood cells. The molecular mechanisms controlling multipotentiality, self-renewal, quiescence and HSC commitment have been extensively studied. However, numerous issues remain to be addressed and important genes regulating these processes remain to be identified.

Acute Myeloid Leukemia (AML), Myeloproliferative neoplasms (MPNs), myelodysplastic syndromes (MDS) and myelodysplastic/myeloproliferative disorders are clonal stem-cell malignant disorders.

Several genetic mutations have been correlated to AML, and four groups are recognized: (i) AMLs with recurrent genetic abnormalities AML t(8; 21)(q22; q22) with RUNX1-ETO fusion gene; AML with abnormal bone marrow eosinophils and inv(16)(p13; q22) or t(16; 16)(p13; q22) with CBFB/MYH11 rearrangement; acute promyelocytic leukaemia APL with t(15; 17)(q22; q12) PML/RARA; AML with 11q23 (MLL) abnormalities); (ii) AML with multilineage dysplasia following MDS or MDS/MPN or without antecedent of MDS or MPN; (iii) AML or MDS therapy related and (iv) other unclassified AMLs, which comprise the group of AMLs with normal karyotype whose prognosis is based on molecular analysis of oncogenes such as mutations of FLT3-ITD or NPM1.

Myelodysplastic/myeloproliferative neoplasms include four myeloid diseases grouped in 1999 by the WHO: chronic myelomonocytic leukemia (CMML), juvenile myelomonocytic leukemia (JMML), atypical chronic myeloid leukemia (aCML) and unclassified myelodysplastic/myeloproliferative syndromes (U-MDS/MPS).

MPNs include chronic myelogenous leukemia (CML), polycythemia vera (PV), essential thrombocytopenia (ET) and idiopathic myelofibrosis (IMF). MPNs are characterized by an increased proliferation of one or several myeloid lineages and are commonly associated with an acquired constitutive kinase activity, as exemplified by the JAK2^{V617F} mutation in Polycythemia Vera.

MDS are classified into several classes including refractory anemia (RA), and refractory cytopenia with multilineage dysplasia (RCMD), and RA with excess of blasts (RAEB). MDS are characterized by ineffective hematopoiesis in one or more of the lineage of the bone marrow, but the underlying molecular defects are still poorly understood. No biological markers, except morphological features, are currently available for early diagnosis and prognosis.

The ASXL (additional sex combs) family of genes has three members in humans encoding poorly characterized proteins containing a C-terminal PHD finger (plant homeodomain). It is now expected that these ASXL proteins regulate chromatin remodeling and are potentially linked to regulation of transcription.

More specifically, the ASXL1 (additional sex combs like 1) gene (also known as KIAA0978; MGC71111; MGC117280) is located on the chromosomal region 20q11, comprises 12 exons over about 100 kb. This gene is referenced under the accession number ID 171023 and its cDNA (Accession number NM_{—}015338, SEQ ID No 1) encodes a protein of 1541 amino acids (Accession number NP_{—}056153, SEQ ID No 2).

The ASXL1 protein helps recruiting polycomb and thrithorax complexes to specific domains and shares four conserved domains consisting in i) the ASXN domain (amino acids 1 to 86), ii) the ASXM (amino acids 250 to 361), iii) the NR box (the amino acids 1107 to 1112) and i) the PHD domain (amino acids 1506 to 1541, SEQ ID No 3). The ASXL1 protein plays a role in transcription regulation of differentiation (e.g. retinoic acid pathway) and self-renewal programs.

### SUMMARY OF THE INVENTION

Inventors report here mutations of ASXL1 gene in myelodysplastic syndromes (MDS), in myelodysplastic/myeloproliferative neoplasms (Le, chronic myelomonocytic leukemia (CMML)), in MPNs, and in Acute Myeloid Leukemia (AML).

The invention relates to a method for diagnosing a myeloid cancer in a subject, which comprises the step of analyzing a biological sample from said subject by determining the presence or the absence of a mutation in the ASXL1 (additional sex combs like 1) gene coding for the polypeptide having the sequence SEQ ID No 2, wherein the presence of such a mutation is correlated with a myeloid cancer. Identification of the presence or the absence of the mutation may be performed by comparison to a normal control, e.g. a cell line, which does not comprise said mutation. The method may further comprise the step of recording the presence or absence of said mutation at a particular position.

Advantageously, said myeloid cancer is selected in the group consisting of myelodysplastic syndrome (MDS), myelodysplatic/myeloproliferative neoplasms, myeloproliferative neoplasm (MPN) and acute myeloid leukemia (AML).

In a first preferred embodiment, said method is for diagnosing a myelodysplastic syndrome (MDS) in a subject.

In a second preferred embodiment, said method is for diagnosing a myelodysplatic/myeloproliferative neoplasm, preferably a chronic myelomonocytic leukemia (CMML) in a subject, and most preferably for differentiating MP-CMML from MD-CMML.

In a third preferred embodiment, said method is for diagnosing a myeloproliferative neoplasm (MPN) in a subject, preferably said MPN is a primary myelofibrosis (PMF), post-polycythemia vera myelofibrosis (post-PV MF) or post essential thrombocythemia myelofibrosis (post-ET MF).

In a fourth preferred embodiment, said method is for diagnosing an acute myeloid leukemia (AML) in a subject, more preferably said AML is a secondary AML and still preferably a secondary AML following a chronic myeloid disease.

Advantageously, said mutation is selected in the group consisting of insertions, deletions, and point mutations corresponding to missense mutation and nonsense mutations, preferably in the group consisting of insertions, deletions, and nonsense mutations.

Preferably, said mutation results in the expression of a mutated ASXL1 protein, said mutated ASXL1 protein does not comprises any longer its Plant HomeoDomain (PHD domain, SEQ ID No 3) or a fragment thereof.

In a second aspect, the invention relates to a kit for diagnosing myeloid cancer in a subject comprising at least one nucleic acid probe or oligonucleotide or at least one antibody, which can be used in a method as defined previously for determining the presence or the absence of a mutation in the ASXL1 (additional sex combs like 1) gene coding for the polypeptide having the sequence SEQ ID No 2, wherein the presence of such a mutation is correlated with a myeloid cancer.

In a third aspect, the present invention provides a method for the prognosis of the outcome of a myeloid cancer in a subject, which comprises the step of analyzing a biological sample from said subject by determining the presence or the absence of a mutation in the ASXL1 (additional sex combs like 1) gene coding for the polypeptide having the sequence SEQ ID No 2, wherein the presence of the mutation is indicative of a poor prognosis of said patient, and the absence of the mutation is suggestive of a good prognosis of said patient. Mutations that are suitable for said prognosis include, but are not limited to, disclosed mutations in the ASXL1 gene. The presence or the absence of the mutation may be performed by comparison to a normal control, e.g. a cell line, which does not comprise said mutation. The method may further comprise the step of recording the presence or absence of said mutation at a particular position.

Advantageously, said myeloid cancer is a MDS and the method of the invention is for the prognosis of the progression of said MDS to refractory anemia, preferably to refractory anemia with excess of blasts type 2 (RAEB). The presence of an ASXL1 mutation, such as Gly646Trp FS, is indicative of a risk of a progression of said MDS to RAEB2.

Still advantageously, said myeloid cancer is a MDS and the method of the invention is for the prognosis of the progression of said MDS to AML, preferably to secondary anemia.

Still advantageously, said myeloid cancer is a CMML and the method of the invention is for the prognosis of the progression of said CMML, preferably MP-CMML, to AML. The presence of an ASXL1 mutation, such as Gly646Trp FS, is indicative of a risk of a progression of said CMML to AML.

Still advantageously, said myeloid cancer is a CMML and the method of the invention is for the prognosis of the outcome for said patient. The presence of an ASXL1 mutation, such as Gly646Trp FS, was associated with a poor outcome.

Still advantageously, said myeloid cancer is a polycythemia vera (PV) and the method of the invention is for the prognosis of the progression of said PV to post-polycythemia vera myelofibrosis (post-PV MF). The presence of an ASXL1 mutation is indicative of a risk of a progression of said PV to Post-PV MF.

Still advantageously, said myeloid cancer is an essential thrombocythemia (ET) and the method of the invention is for the prognosis of the progression of said PV to post-essential thrombocythemia myelofibrosis (post-ET MF). The presence of an ASXL1 mutation is indicative of a risk of a progression of said ET to Post-PV ET.

In a fourth aspect, the present invention provides a method for predicting the response to a treatment for a myeloid cancer in a subject, which comprises the step of analyzing a biological sample from said subject by determining the presence or the absence of a mutation in the ASXL1 (additional sex combs like 1) gene coding for the polypeptide having the sequence SEQ ID No 2. The method may further comprise the step of recording the presence or absence of said mutation at a particular position. Mutations that are suitable for said predicting include, but are not limited to, mutations in the ASXL1 gene, said mutations leading potentially to epigenetic modifications. Preferably, the detection of ASXL1 mutation(s) results in a good prognostic for treatment using one or more drugs that may be selected within the group consisting in demethylating agents and HDAC (Histone deacetylases) inhibitors or a combination thereof. Demethylating agents may include Cytidine analogs, such as 5-azacytidine (azacitidine VIDAZA) and 5-azadeoxycytidine (decitabine, DACOGEN), e.g. for the treatment of Myelodysplastic syndrome (MDS), or Procaine. HDAC inhibitors may include ITF2357 (Givinovas, ITALFARMACO), Valproic Acid, Panobinostat. Romidepsin, Vorinostat.

### BRIEF DESCRIPTION OF THE DRAWINGS

FIG. 1 shows the representation of the ASXL1 protein with known motifs and domains.

Table I shows the pairs of primers sequences used to amplify and sequence the ASXL1 gene.

Table II shows the molecular features of 40 studied MDS cases.

Table III shows the mutations of ASXL1 in CMML.

Table IV shows the clinical and molecular data of 112 MPNs patients with ASXL1 mutations.

Table V shows the clinical and biological features of ASXL1 mutated and unmutated CMML patients.

FIG. 2 shows the Kaplan-Meier overall survival curves of CMML patients according to ASXL1 mutational status.

FIG. 3 shows the Kaplan-Meier overall survival curves of CMML patients according to ASXL1 mutational status in patients whose disease had not evolved in acute leukaemia.

### DETAILED DESCRIPTION

The present invention is based on the discovery by the present inventors that the ASXL1 gene is often targeted by mutations and/or deletions in tumoral cells in patients sufferings from MDS, myelodysplatic/myeloproliferative neoplasms, MPN or AML.

Consequently, in one aspect the present invention relates to a method for diagnosing a myeloid cancer in a subject, which comprises the step of analyzing a biological sample from said subject by determining the presence or the absence of a mutation in the ASXL1 (additional sex combs like 1) gene coding for the polypeptide having the sequence SEQ ID No 2, wherein the presence of such a mutation is correlated with a myeloid cancer.

As used herein, the term “subject” refers to a mammal, preferably a human.

Said subject may be healthy, but the method of the invention is particularly useful for testing a subject thought to develop or to be predisposed to developing a myeloid cancer. In that case, the method of the invention enables to confirm that said subject develops or is predisposed for developing a myeloid cancer.

Preferably, said myeloid cancer is selected in the group consisting of myelodysplastic syndrome (MDS), myelodysplatic/myeloproliferative neoplasms, myeloproliferative neoplasm (MPN) and acute myeloid leukemia (AML).

In a first preferred embodiment, said method is for diagnosing a myelodysplastic syndrome (MDS) in a subject, preferably a Refractory anemia with excess of blasts type 2 (RAEB2).

In fact, the inventors have established on a MDS patients panel that at least 11% of them comprise an ASXL1 mutation. In Refractory anemia with excess of blasts type 2 (RAEB2) the inventors have established that at least 47% of them display an ASXL1 mutation.

Preferably, the ASXL1 mutation is Gly646Trp FS.

In a second preferred embodiment, said method is for diagnosing a myelodysplatic/myeloproliferative disorders, preferably a chronic myelomonocytic leukemia (CMML) in a subject.

In fact, the inventors have established on a CMML patients panel that at least 43% of them comprise an ASXL1 mutation.

Still preferably, said CMML is a myeloproliferative form of CMML (MP CMML).

Since, the inventors have established on a MP CMML patients panel that at least 62% of them comprise an ASXL1 mutation.

Advantageously, the method of the invention is for differentiating MP-CMML from MD-CMML since ASXL1 mutations are more rare in MD CMML patients.

In a third preferred embodiment, said method is for diagnosing a myeloproliferative neoplasm (MPN) in a subject.

In fact, the inventors have established on a MPN patients panel that at least 8% of them comprise an ASXL1 mutation.

Preferably, said MPN is a primary myelofibrosis (PMF) since the inventors have established on a PMF patients panel that at least 33% of them comprise an ASXL1 mutation.

Still preferably, said MPN is post-polycythemia vera myelofibrosis (post-PV MF) since the inventors have established that at least 66% of them comprised an ASXL1 mutation.

Still preferably, said MPN is post-essential thrombocythemia myelofibrosis (post-ET MF) since the inventors have established that at least 25% of them comprised an ASXL1 mutation.

Still preferably, said MPN is essential thrombocythemia (ET) and the presence of a mutation in the ASXL1 gene exclude a reactive thrombocytosis (RT) since the inventors have established that reactive thrombocytosis patients do not share any ASXL1 mutation.

In a fourth preferred embodiment, said method is for diagnosing an acute myeloid leukemia (AML) in a subject.

In fact, the inventors have established on an AML patients panel that at least 19% of them comprise an ASXL1 mutation.

Still preferably, said AML is a secondary AML and more preferably a secondary AML following a chronic myeloid disease. In fact, the inventors have established on a secondary AML patients panel that at least 53% of them comprise an ASXL1 mutation. Moreover 75% of the secondary AML patients following a chronic myeloid disease panel comprise an ASXL1 mutation

As used herein, the expression “biological sample” refers to solid tissues such as, for example, a lung biopsy; buccal swab, fluids and excretions such as for example, sputum, induced sputum, blood, serum, plasma, urine. Preferably, said biological sample is a blood or bone marrow sample, preferably a bone marrow sample. Preferably, only a biological sample containing cells including genomic DNA (or optionally RNA) from the subject to be tested is required.

As used herein, the term “mutation” corresponds to any modification in the sequence of the original nucleic acid sequence. These mutations comprise small-scale mutations, or large scale mutations. Small scale mutations are those affecting a gene in one or a few nucleotides, including point mutations, insertions or deletions of one or more extra nucleotides in the DNA. Point mutations can be silent, missense and nonsense mutation. Large scale mutation in the genomic structure, such as gene duplications, deletions, or mutations whose effect is to juxtapose previously separate pieces of DNA, potentially bringing together separate genes to form functionally distinct fusion genes.

Preferably, said mutation is selected in the group consisting of insertions, deletions, and point mutations corresponding to missense mutation and nonsense mutations.

More preferably, said mutation is selected in the group consisting of insertions, deletions, and nonsense mutations.

Moreover, the inventors have established that the exon 12 of the gene encoding the PHD domain of the ASXL1 protein is preferentially targeted by the deleterious mutations in the studied patients (See examples).

Thus, and still preferably, said mutation results in the expression of a mutated ASXL1 protein that does not comprise any PHD domain (SEQ ID No 3) or a fragment thereof.

Said mutated protein can result from the introduction of a non sense mutation leading to the introduction of a stop codon (X) in the open reading frame of the ASXL1 protein. As an example, said non-sense mutation is selected in the group comprising Tyr591X, Gln592X, Lys618X, Arg693X, Gln759X, Gln768X, Leu775X and Arg1068X.

Said mutated protein can also result from a frame-shift (FS) because of an insertion of a deletion in the ASXL1 gene. As an example, said insertion or deletion is selected in the group of the ones inducing the expression of the mutated ASXL1 protein with the following mutations Gly64Trp FS, Arg596Pro FS, Ala611Arg FS, His630Pro FS, Gly646Trp FS, Leu762Phe FS, Trp796Gly FS, Thr822Asn FS, Thr836Leu FS, Ser846Gln FS, Asp879Glu FS, Lys888Glu FS, Leu1213Ile FS, Pro1263Gln FS, Leu1266H is FS, Trp1271Lys FS, or Ser1457Pro FS.

In another preferred embodiment of the invention, the determining step is done on genomic DNA.

Typical techniques for detecting the presence of a mutation in DNA may include restriction fragment length polymorphism, hybridization techniques, DNA sequencing, exonuclease resistance, microsequencing, solid phase extension using ddNTPs, extension in solution using ddNTPs, oligonucleotide ligation assays, methods for detecting single nucleotide polymorphisms such as dynamic allele-specific hybridization, ligation chain reaction, mini-sequencing, DNA “chips”, high resolution melting (HRM) method, amplification-refractory mutation system (ARMS) method, allele-specific oligonucleotide hybridization with single or dual-labelled probes merged with PCR or with molecular beacons, Scorpions® probes (DxS Genotyping), MGB® (Minor Groove Binding) probes (NANOGEN) and others.

Advantageously, the mutation is detected on the cDNA of the ASXL1 gene by either PCR and sequencing, SNP-array or CGH, all of them being well known for the skilled person.

Comparative genomic hybridization (CGH) is a molecular cytogenetic method of screening a tumor for genetic changes. The alterations are classified as DNA gains and losses and reveal a characteristic pattern that includes mutations at chromosomal and subchromosomal levels. The method is based on the hybridization of fluorescently labeled tumor DNA (frequently fluorescein (FITC)) and normal DNA (frequently rhodamine or Texas Red) to normal human metaphase preparations. Using epifluorescence microscopy and quantitative image analysis, regional differences in the fluorescence ratio of gains/losses vs. control DNA can be detected and used for identifying abnormal regions in the genome. CGH will detect only unbalanced chromosomes changes. Structural chromosome aberrations such as balanced reciprocal translocations or inversions can usually not be detected, as they do not systematically change the copy number.

In another preferred embodiment of the invention, the determining step is realized on ASXL1 mRNA/cDNA.

Such analysis can be assessed by preparing mRNA/cDNA from cells in a biological sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TAQMAN), and probes arrays such as GENECHIP DNA Arrays (AFFYMETRIX).

In still another preferred embodiment of the invention, the determining step is realized on the ASXL1 protein.

Such analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the ASXL1 gene (SEQ ID No 2), and preferably to the PHD domain (SEQ ID No 3) of the ASXL1 protein.

Said analysis can be assessed by a variety of techniques well known by one of skill in the art including, but not limited to, enzyme immunoassay (EIA), radioimmunoassay (RIA), Western blot analysis and enzyme linked immunoabsorbent assay (ELISA).

Polyclonal antibodies can be prepared by immunizing a suitable animal, such as mouse, rabbit or goat, with the ASXL1 protein (SEQ ID No 2) or the PHD domain thereof (SEQ ID No 3).The antibody titer in the immunized animal can be monitored over time by standard techniques, such as with an ELISA using immobilized polypeptide. At an appropriate time after immunization, e.g., when the specific antibody titers are highest, antibody producing cells can be obtained from the animal and used to prepare monoclonal antibodies (mAb) by standard techniques.

The skilled person can also use commercially available ASXL1 monoclonal antibodies, such as the monoclonal antibodies commercialized by ABCAM or by SANTA CRUZ BIOTECHNOLOGY Inc.

In a second aspect, the present invention refers to a kit for diagnosing myeloid cancer in a subject comprising at least one nucleic acid probe or oligonucleotide or at least one antibody, which can be used in a method as defined previously, for determining the presence or the absence of a mutation in the ASXL1 (additional sex combs like 1) gene coding for the polypeptide having the sequence SEQ ID No 2, wherein the presence of such a mutation is correlated with a myeloid cancer.

Preferably, the oligonucleotide is at least one PCR primer, preferably a set of PCR primers is provided, which allows to amplify the ASXL1 gene or a fragment thereof. The skilled person readily provides such an oligonucleotide or set of PCR primers which allows to amplify a region of the ASXL1 gene, provided that the nucleic acid sequence of the ASXL1 gene is well known (Accession number NC_{—}000020.10, nucleotides 30,946,153 to 31,027,122; and accession number NM_{—}015338 for the corresponding cDNA, SEQ ID No 1).

As an example, said pairs of PCR primers are selected in the group comprising SEQ ID No 4 and SEQ ID No 5, SEQ ID No 6 and SEQ ID No 7, SEQ ID No 8 and SEQ ID No 9, SEQ ID No 10 and SEQ ID No 11, SEQ ID No 12 and SEQ ID No 13, SEQ ID No 14 and SEQ ID No 15, SEQ ID No 16 and SEQ ID No 17, SEQ ID No 18 and SEQ ID No 19, SEQ ID No 20 and SEQ ID No 21, SEQ ID No 22 and SEQ ID No 23, SEQ ID No 24 and SEQ ID No 25, SEQ ID No 26 and SEQ ID No 27, SEQ ID No 28 and SEQ ID No 29, SEQ ID No 30 and SEQ ID No 31, and SEQ ID No 32 and SEQ ID No 33. Such primers are disclosed in table I.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte Specific Reagents (ASRs) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

The present kits can also include one or more reagents, buffers, hybridization media, nucleic acids, primers, nucleotides, probes, molecular weight markers, enzymes, solid supports, databases, computer programs for calculating dispensation orders and/or disposable lab equipment, such as multi-well plates, in order to readily facilitate implementation of the present methods. Enzymes that can be included in the present kits include nucleotide polymerases and the like. Solid supports can include beads and the like whereas molecular weight markers can include conjugatable markers, for example biotin and streptavidin or the like.

In one embodiment, the kit is made up of instructions for carrying out the method described herein for diagnosing a myeloid cancer in a subject. The instructions can be provided in any intelligible form through a tangible medium, such as printed on paper, computer readable media, or the like.

In another embodiment, said myeloid cancer is selected in the group consisting of myelodysplastic syndromes (MDS), myelodysplatic/myeloproliferative neoplasms, myeloproliferative neoplasms (MPN) and acute myeloid leukemias (AML).

In a third aspect, the present invention provides a method for the prognosis of the outcome of a myeloid cancer in a subject, which comprises the step of analyzing a biological sample from said subject by determining the presence or the absence of a mutation in the ASXL1 (additional sex combs like 1) gene coding for the polypeptide having the sequence SEQ ID No 2, wherein the presence of the mutation is indicative of a poor prognosis of said patient, and the absence of the mutation is suggestive of a good prognosis of said patient. Mutations that are suitable for said prognosis include, but are not limited to, disclosed mutations in the ASXL1 gene. The presence or the absence of the mutation may be performed by comparison to a normal control, e.g. a cell line, which does not comprise said mutation. The method may further comprise the step of recording the presence or absence of said mutation at a particular position.

Advantageously, said myeloid cancer is a MDS and the method of the invention is for the prognosis of the progression of said MDS to refractory anemia, preferably to refractory anemia with excess of blasts type 2 (RAEB). The presence of an ASXL1 mutation, such as Gly646Trp FS, is indicative of a risk of a progression of said MDS to RAEB2.

Still advantageously, said myeloid cancer is a MDS and the method of the invention is for the prognosis of the progression of said MDS to AML, preferably to secondary anemia.

Still advantageously, said myeloid cancer is a CMML and the method of the invention is for the prognosis of the progression of said CMML, preferably MP-CMML, to AML. The presence of an ASXL1 mutation, such as Gly646Trp FS, is indicative of a risk of a progression of said CMML to AML. In fact, the inventors have determined that among the CMML patients with no mutation in the ASXL1 gene, none of them has evolved to AML.

Still advantageously, said myeloid cancer is a CMML and the method of the invention is for the prognosis of the outcome for said patient. The presence of an ASXL1 mutation, such as Gly646Trp FS, was associated with a poor outcome.

Still advantageously, said myeloid cancer is a polycythemia vera (PV) and the method of the invention is for the prognosis of the progression of said PV to post-polycythemia vera myelofibrosis (post-PV MF). The presence of an ASXL1 mutation is indicative of a risk of a progression of said PV to Post-PV MF.

Still advantageously, said myeloid cancer is an essential thrombocythemia (ET) and the method of the invention is for the prognosis of the progression of said PV to post-essential thrombocythemia myelofibrosis (post-ET MF). The presence of an ASXL1 mutation is indicative of a risk of a progression of said ET to Post-PV ET.

In a fourth aspect, the present invention provides a method for predicting the response to a treatment for a myeloid cancer in a subject, which comprises the step of analyzing a biological sample from said subject by determining the presence or the absence of a mutation in the ASXL1 (additional sex combs like 1) gene coding for the polypeptide having the sequence SEQ ID No 2. The method may further comprise the step of recording the presence or absence of said mutation at a particular position. Mutations that are suitable for said predicting include, but are not limited to, mutations in the ASXL1 gene, said mutations leading potentially to epigenetic modifications. Preferably, the detection of ASXL1 mutation(s) results in a good prognostic for treatment using one or more drugs that may be selected within the group consisting in demethylating agents and HDAC (Histone deacetylases) inhibitors or a combination thereof. Demethylating agents may include Cytidine analogs, such as 5-azacytidine (azacitidine VIDAZA) and 5-azadeoxycytidine (decitabine, DACOGEN), e.g. for the treatment of Myelodysplastic syndrome (MDS), or Procaine. HDAC inhibitors may include ITF2357 (Givinovas, ITALFARMACO), Valproic Acid, Panobinostat. Romidepsin, Vorinostat.

In the following, the invention is described in more detail with reference to amino acid sequences, nucleic acid sequences and the examples. Yet, no limitation of the invention is intended by the details of the examples. Rather, the invention pertains to any embodiment which comprises details which are not explicitly mentioned in the examples herein, but which the skilled person finds without undue effort.

### EXAMPLES

### 1) Alteration of the ASXL1 Gene in Patients Suffering from MDS

Three Types of aCHG Profiles in MDSs

A series of bone marrow (BM) samples were collected from patients with MDS, with AML with multilineage dysplasia (AML-MLD), and with AML secondary to CMML.

According to the French-American-British (FAB) and WHO criteria, the MDS panel comprised three RA, nine RARS (including one with idiopathic myelofibrosis), three RCMD (including two with ring sideroblasts), 10 RAEB1, eight RAEB2 and two MDS-U.

The majority of MDS samples were collected at the time of diagnosis; some were in therapeutic abstention of a known MDS and some were under symptomatic treatment. All were de novo except two cases secondary to treatment for solid tumours.

Genome-wide high-density arrays were used to study the aCGH profiles of 40 MDS/AML samples from 38 patients.

The DNA was extracted by the ALLPREP DNA/RNA isolation kit MACHEREY NAGEL from total bone marrow cells, as recommended by the supplier.

DNA imbalances were analysed by Array comparative genomic hybridization (aCGH) using 244K CGH MICROARRAYS (Hu-244A; AGILENT TECHNOLOGIES) an the résolution was up to 6 kb. Scanning was done with Agilent Autofocus Dynamic Scanner (G2565BA; AGILENT TECHNOLOGIES). Data analysis was made as previously described in GELSI-BOYER et al. (BMC Cancer, vol. 8, p:299-314, 2008) and visualized with CGH ANALYTICS 3.4 software (AGILENT TECHNOLOGIES). Extraction data (log₂ ratio) was done with CGH analytics while the normalized and filtered log₂ ratios were obtained from ‘FEATURE EXTRACTION’ software (AGILENT TECHNOLOGIES). Copy number changes were characterized as reported in GELSI-BOYER et al (abovementioned, 2008).

The results are summarized in Table II.

In these results, tree main types of profiles were observed.

Type 1 profiles showed gains or losses that were already visible on the karyotype and affected large regions of the genome, such as trisomy 8, deletions of part of the 5q and 20q arms, or deletion or complex rearrangements of chromosome 7. Deletions on 5q arm were quite large and always comprised RPS14, HSPA9B and many other genes, including CXXC5 (CXXC finger 5).

Type 2 profiles showed rare and limited gains or losses that affected few genes. On case (i.e. case 190) showed several regions with small deletions. One of these at 20q11 contained the ASXL1 (additional sex combs 1) and DNMT3B (DNA cytosine-5-methyltransferase 3 beta) genes and another one at 2p23 the ASXL2 and DNMT3A paralogous genes. The same case also showed a deletion of BAZ2B on chromosome arm 2q. Gains were also observed (i.e. MAP3K4 in case 167) but less frequently than deletions.

In cases with a type 3 profile no genomic copy number aberration (CNA) could be detected. This profile was found in 22/40 cases (55%). We have indicated this by ‘no CNA’ in Table I.

Mutations of Candidate Genes in MDSs

We analysed the sequences of several candidate genes in our MDS samples.

Somatic mutations of HRAS, KRAS, NRAS, RUNX1, NFIA, CTNNB1, TET2 and ASXL1 genes were searched by sequencing exons and consensus splicing sites after polymerase chain reaction (PCR) amplification of genomic DNA (See Table I for ASXL1). PCR amplifications were done in a total volume of 25 μl PCR mix containing at least 5 ng template DNA, Taq buffer, 200 μmol of each deoxynucleotide triphosphate, 20 pmol of each primer and 1 unit of HOT STAR TAQ (QIAGEN).

PCR amplification conditions were as follows: 95° C. 10 min; 95° C. 30 s, 55° C. 30 s, 72° C. 30 s to 1 min depending on PCR product length for 35 cycles; 72° C. 10 min.

PCR products were purified using MILLIPORE PLATE MSNUO30 (MILLIPORE SAS). Aliquots (1 μl) of the purified PCR products were used for sequencing using the BIG DYE TERMINATOR V1.1 kit (APPLIED BIOSYSTEMS) including the forward or reverse primer.

After G50 purification, sequences were loaded on an ABI 3130XL AUTOMAT (APPLIED BIOSYSTEMS). The sequence data files were analysed using the SEQSCAPE software and all mutations were confirmed on an independent PCR product.

The results are presented in Table I and show that no mutation of the three RAS genes, of the NFIA gene or of the CTNNB1 gene was found.

RUNX1 mutation was found in one case out of 24 tested.

We found several cases mutated for TET2 (to be reported in detail elsewhere). Mutations and deletions of this gene have been discovered recently in about 15-20% of various myeloid diseases including MDSs.

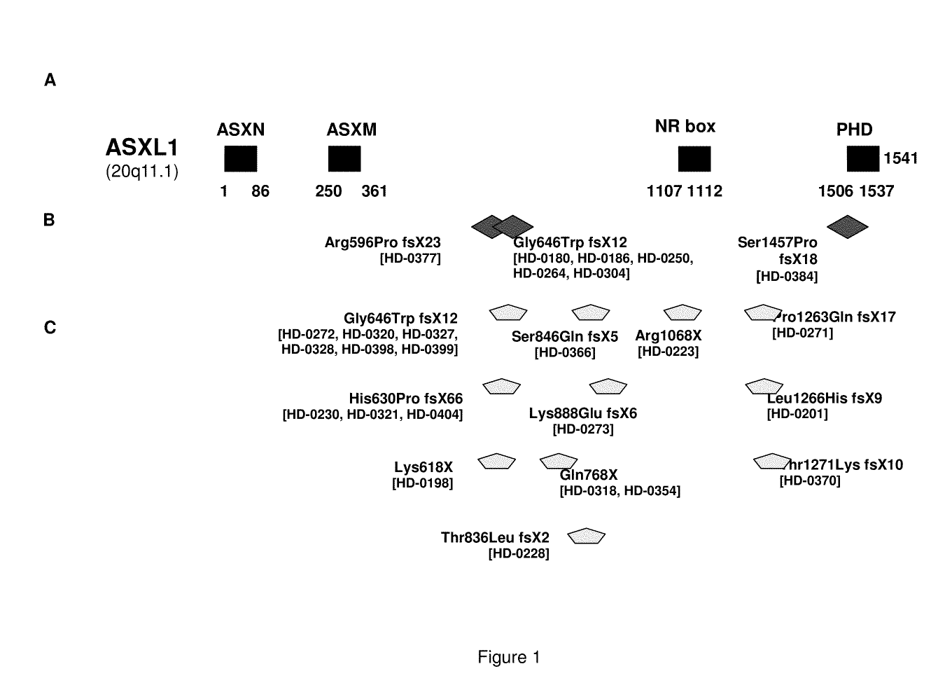

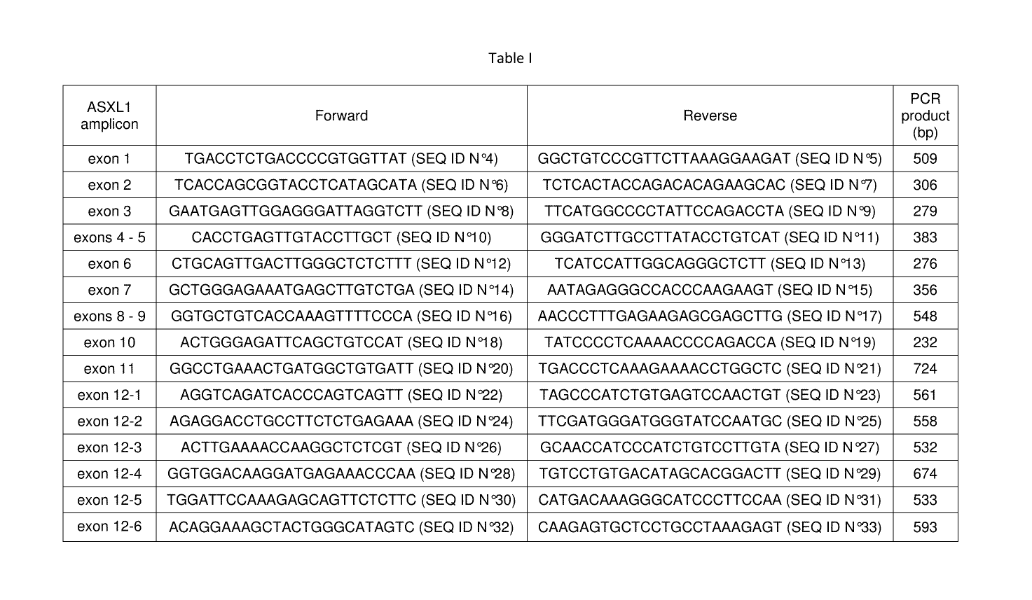

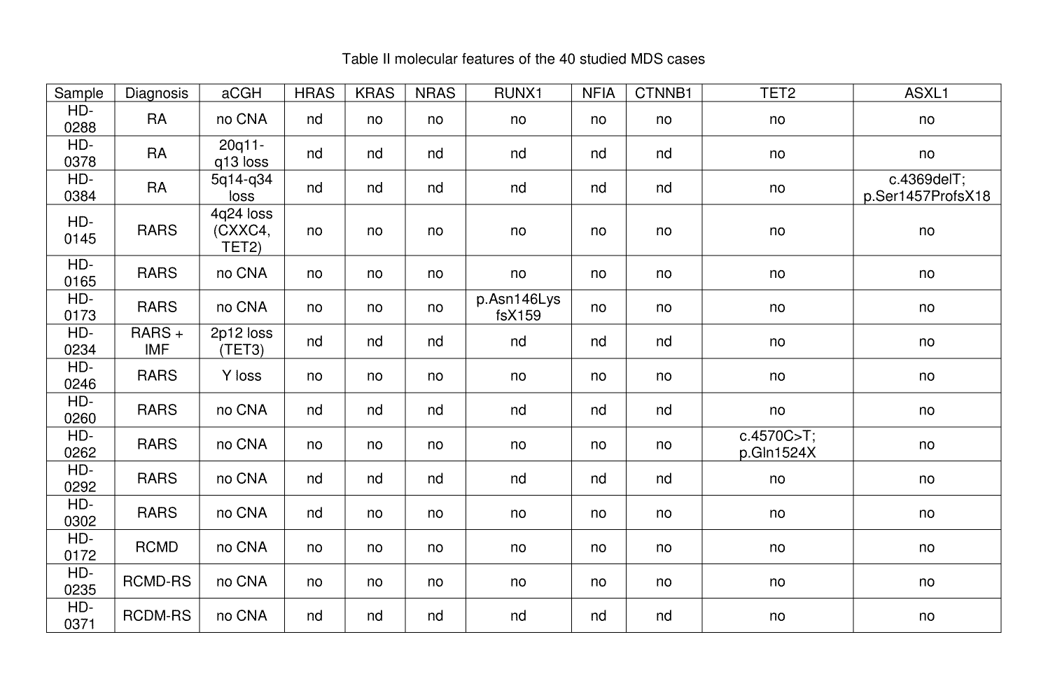

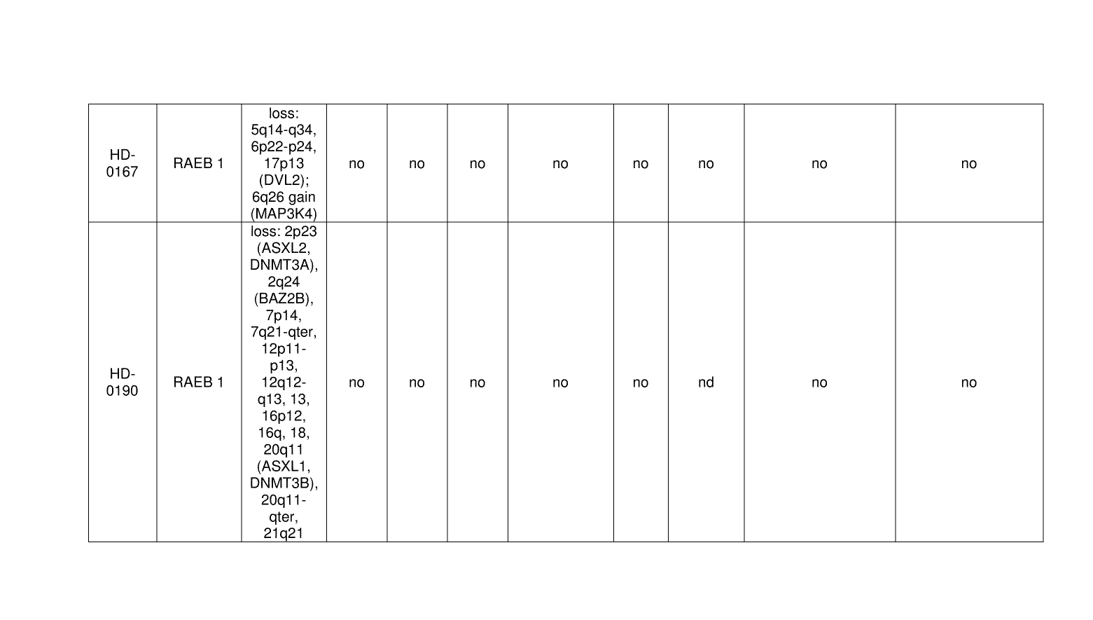

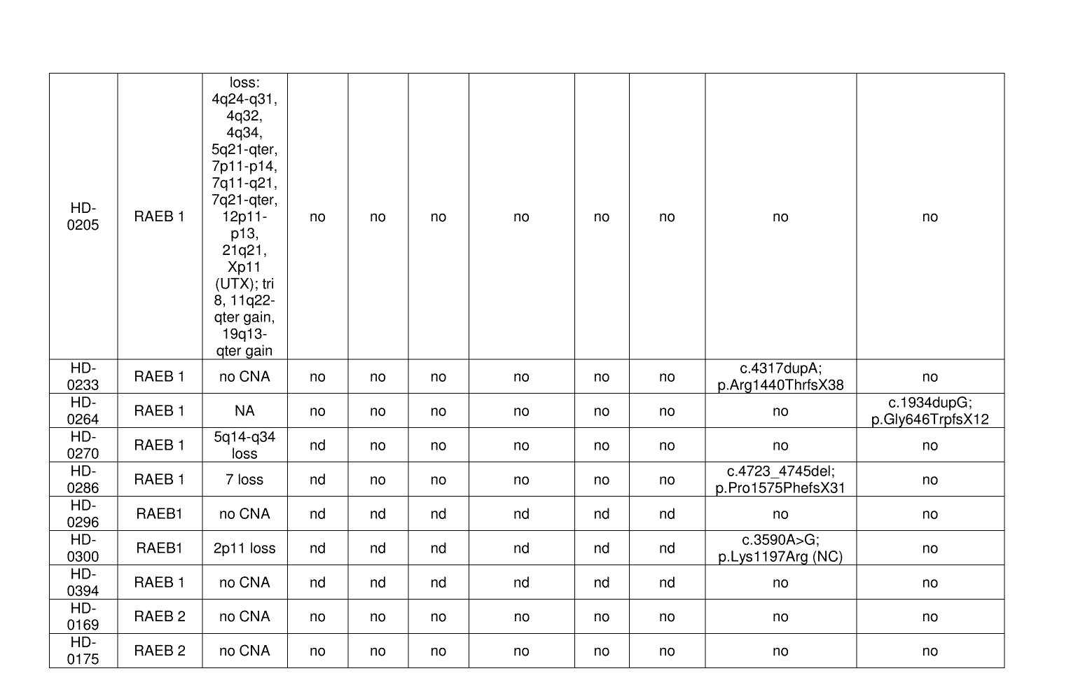

We searched for mutations in the ASXL1 gene, one allele of which was deleted in case 190. We found six mutations in five patients (one of these mutations was found in both the MDS and transformed states) (Table II). The mutations were caused by deletion or duplication of a nucleotide. FIGS. 1A and B shows a schematic representation of the ASXL1 protein with the deduced localization of the mutations. The mutations were all found in exon 12 of the gene and should lead to the truncation of the C-terminus of the protein, which contains a PHD finger.

Finally, e thus found mutations in the ASXL1 gene in 11% of MDS patients.

### 2) Alteration of the ASXL1 Gene in Patients Suffering from Myelodysplastic/Myeloproliferative Disorders

Mutation of ASXL1 in CMML

To determine whether ASXL1 mutations can be found outside MDSs we analysed as described previously the ASXL1 sequence in the bone marrow samples of patients suffering from CMML, a related disease.

According to the FAB and WHO criteria, the series of CMML comprised 21 myeloproliferative (MP-CMML), 18 myelodysplastic (MD-CMML) forms and seven acutely-transformed CMMLs (AT-CMML). All the patients signed an informed consent. The project and collection of samples were reviewed by the independent scientific review board of the Paoli-Calmettes Institute, in accordance with current regulations and ethical concerns.

A total of 19 mutations were found in 44 patients (46 cases) (43%) (Table III). The localization and nature of these mutations are shown in FIG. 1C.

Like in MDS, all the mutations were found in exon 12 and were deletions, duplications, insertions or substitutions of nucleotides. We found 13 mutations in 21 MP-CMML (62%), four in 18 MD-CMML (22%) and two in seven AT-CMML (28%) cases, the difference between MP and MD cases being significant.

Correlations Between ASXL1 Mutation and Clinical and Biological Features in CMML

A series of consecutive bone marrow samples obtained from 53 patients, who all signed an informed consent, were collected. Among these 31 were MP-CMML and 22 were MD-CMML as initially defined by the FAB group with a leukocyte count superior or inferior to 13G/L, respectively. A normal karyotype was observed in 40 patients (20 MP-CMML and 20 MD-CMML); a del(20q)(q11; q13) was found in 3 patients (2 MP-CMML and 1 MD-CMML); a trisomy of a commonly affected chromosomes (8, 19, 21) was encountered in 4 MP-CMML. One MD-CMML had an 11q inversion, one MP-CMML had a t(10; 11)(p12; p15) and one MP-CMML had a t(1; 3)(p36; q21).

The aCGH profiles of 51 of the 53 CMML cases were established as described previously and revealed alterations that were observed by conventional cytogenetics (9/51) except for 3 patients with balanced translocations (HD-0201, HD-0316, HD-0178), and for case HD-0367 with a del(20)(q11q13) for which aCGH did not show a frank deletion at 20q probably due to the low number of affected cells.

For nine cases (17%) aCGH detected rare and limited losses or gains, not visible on the karyotype. They affected very few genes including some with known tumor suppressor function and leukaemogenic activity (NF1, RB1 and TET2). Finally and in 70% of cases (36/51) no copy number aberrations were observed.

The results show also that the genomic alterations detected by conventional cytogenetics or aCGH were different in MP- and MD-CMMLs with 15 alterations out of 31 MP-CMMLs and 4 alterations out of 22 MD-CMMLs. Thus, MP-CMMLs had more genomic alterations than MD-CMMLs (p=0.049).

We studied coding sequences of 13 genes on the 53 cases. In 25 cases (49%) we found 20 frameshift (including 7 times the same p.Gly646Trpfsx12) and 5 nonsense mutations in ASXL1 exon 12. CBL exon 8 mutations were found in 10% of cases (5/47). One case (HD-0223) had a homozygous deletion. One case (HD-0367) had an internal tandem duplication of FLT3. We found 5 IDH mutations in 48 cases (10%); all were in IDH2 (4 times the same p.Arg140Gln). Seven patients out of the 53 cases had a K or NRAS mutation (13%). Twelve out of 53 patients (21%) were mutated for RUNX1 and 36% of patients were mutated for TET2. No mutation was found in NPM1, JAK2 and WT1.

We then studied the prevalence of the mutated genes in MP and MD-CMML. Nineteen of the 25 ASXL1 mutations were found in 30 MP-CMML vs. 6 in 22 MD-CMML. Mutations in ASXL1 but not in RUNX1 or TET2 were more frequent in MP than in MD-CMMLs (p=0.03). No difference was observed between the two forms for CBL, FLT3, IDH1/2, PTPN11, RAS, RUNX1 or TET2. Overall the number of mutations (ASXL1 and proliferation genes) was higher in MP (69/273 events) than in MD-CMMLs (26/172) (p=0.0018).

Since the classification of CMML has always been a matter of debate, thus the ASXL1 mutation corresponds to a molecular basis to the separation of CMML in MP and MD forms initially defined by the FAB group.

In the present study ASXL1 appeared as the most frequently mutated gene in CMML, as it is in MDSs.

The main clinical and biological features of 51 CMML cases were examined with respect to ASXL1 mutations (Table V).

The results shown that the presence of an ASXL1 mutation was associated with higher WBC (30 g/L vs. 15 g/L) (p=0.006), higher blood (p=0.005) and bone marrow monocytosis (p=0.04) and with lower level of blood haemoglobin (p=0.03). No difference was noted in mean cell volume, blood count of neutrophils and platelets, or bone marrow blasts. In MP-CMML ASXL1 mutation correlated with a lower level of haemoglobin (p=0.03) and platelet count (p=0.002) and with a higher monocytosis (p=0.04). In MD-CMML, no correlations with ASXL1 mutation were observed.

Among ASXL1 mutated cases (25/51), eleven (9 MP and 2 MD) had evolved to acute transformation (Table V), whereas no acute transformation was observed in the unmutated cases. In other words, all transformed cases had an ASXL1 mutation but not all ASXL1 mutated cases had progressed to AML.

ASXL1 mutation and acute transformation were thus correlated (p=0.0005). If some mutated cases had not progressed to acute phase, this may be because patients had died before experiencing acute leukaemia.

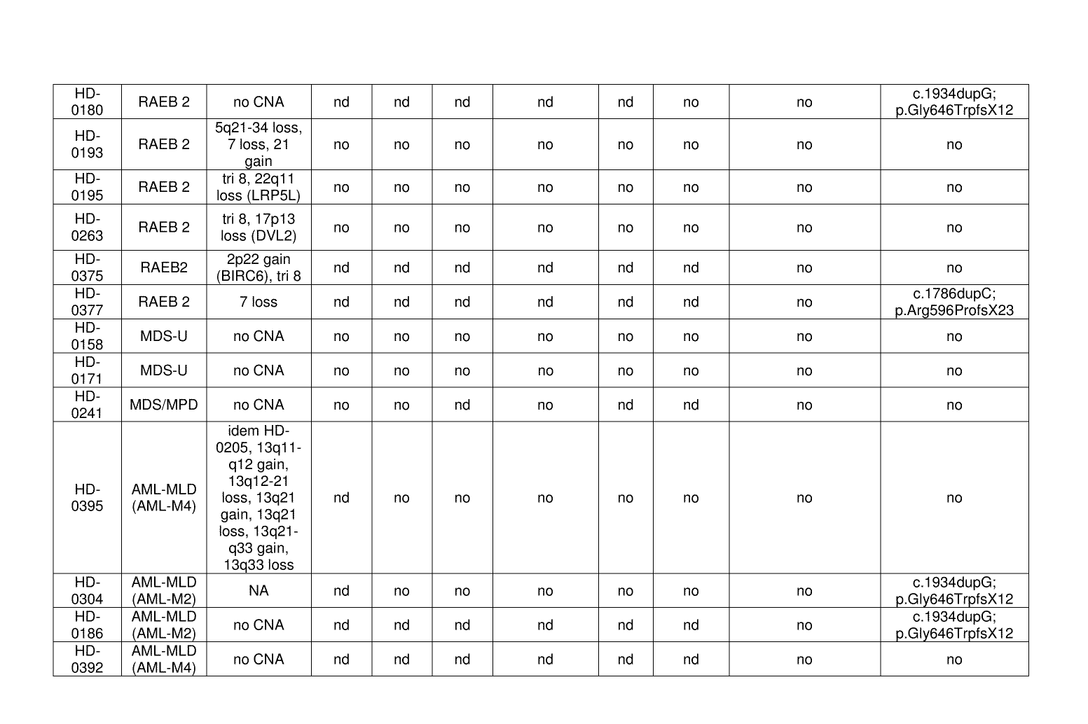

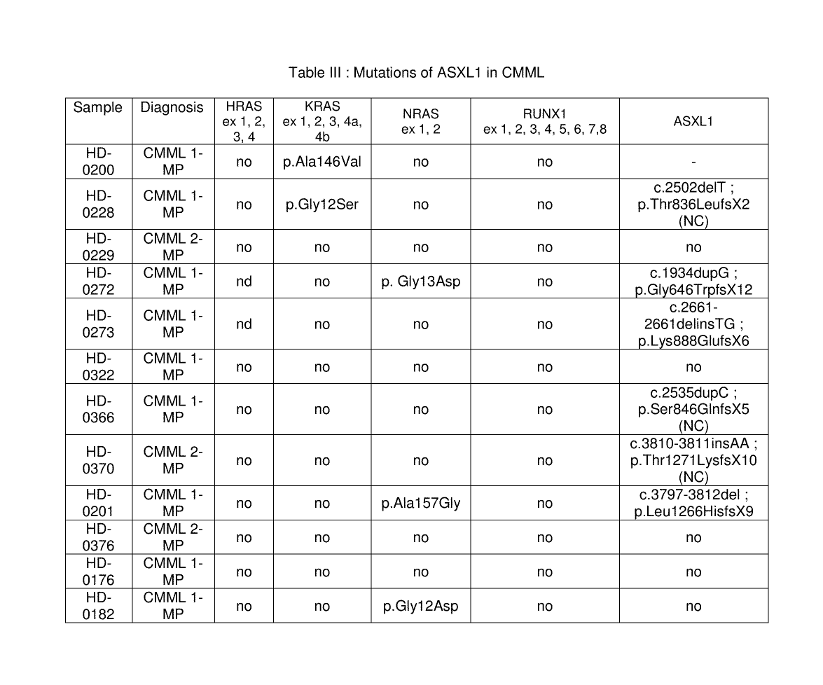

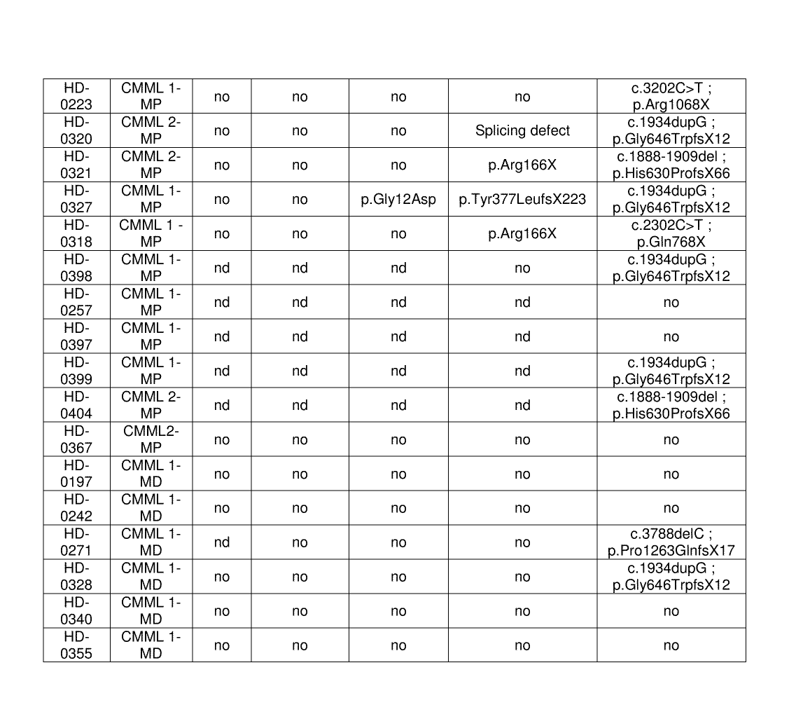

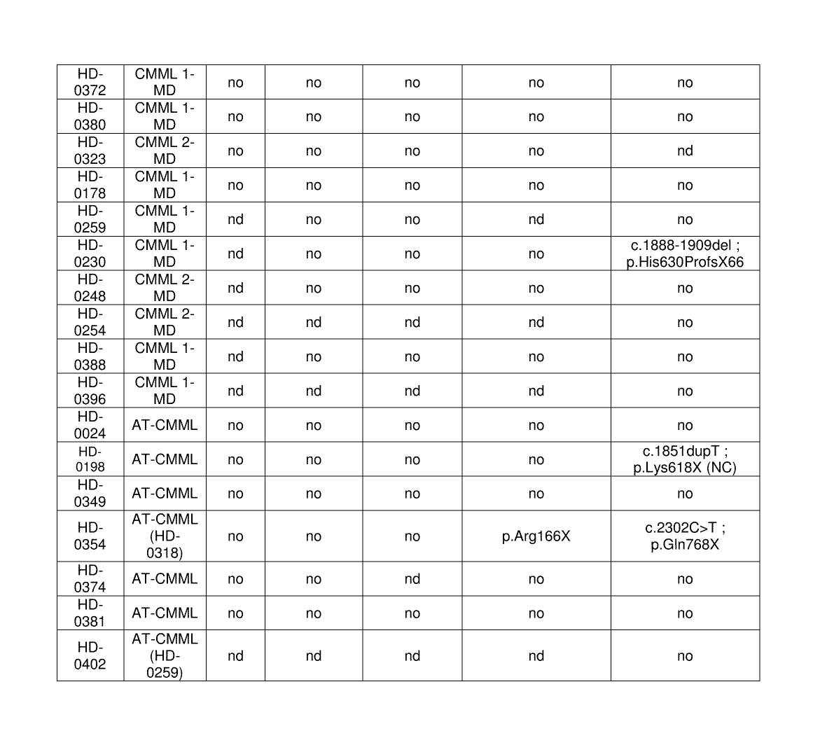

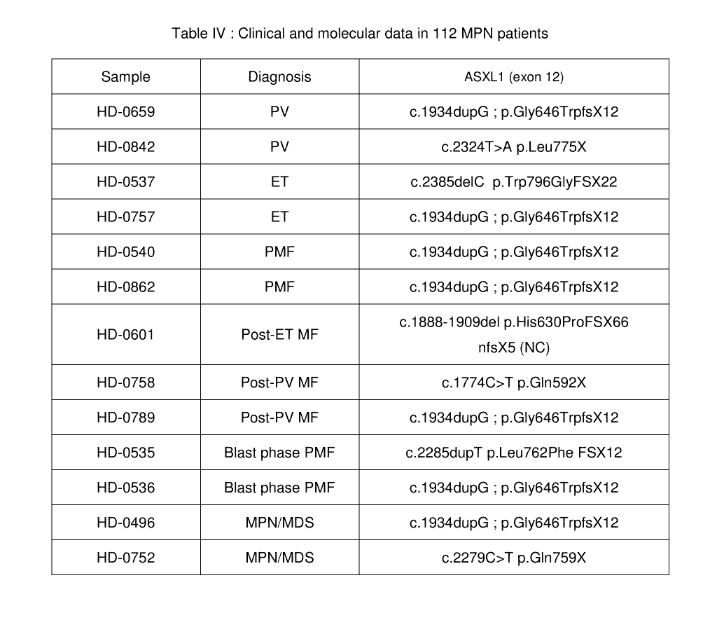

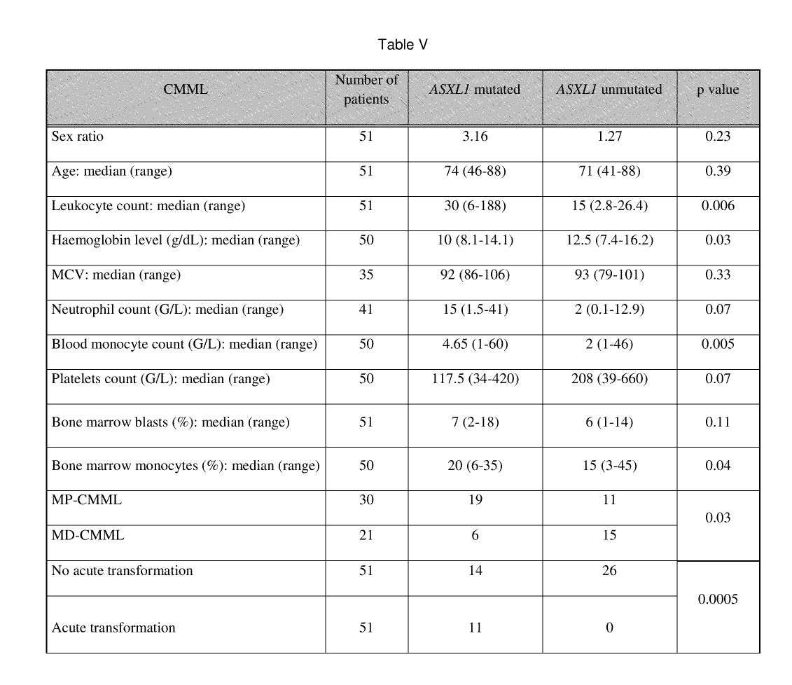

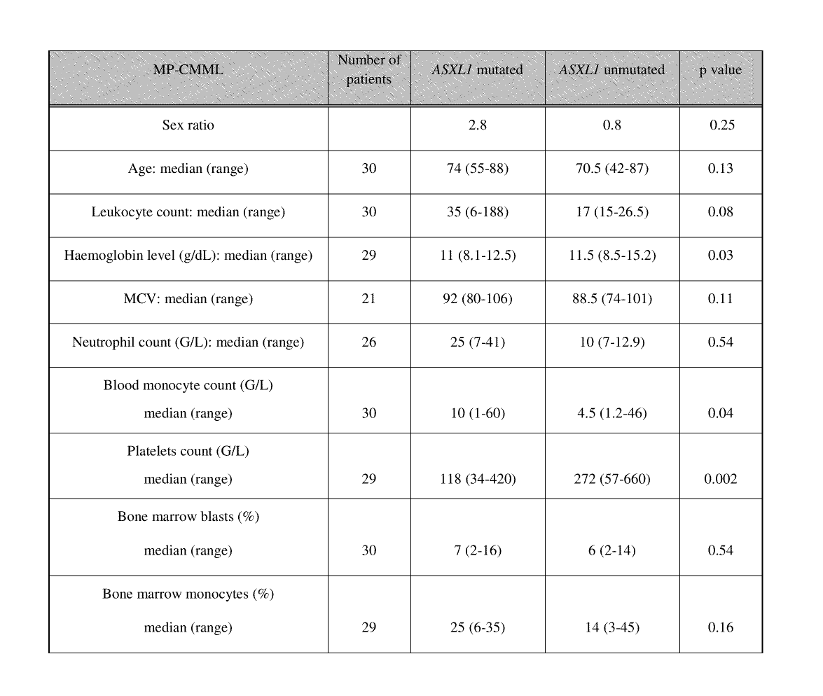

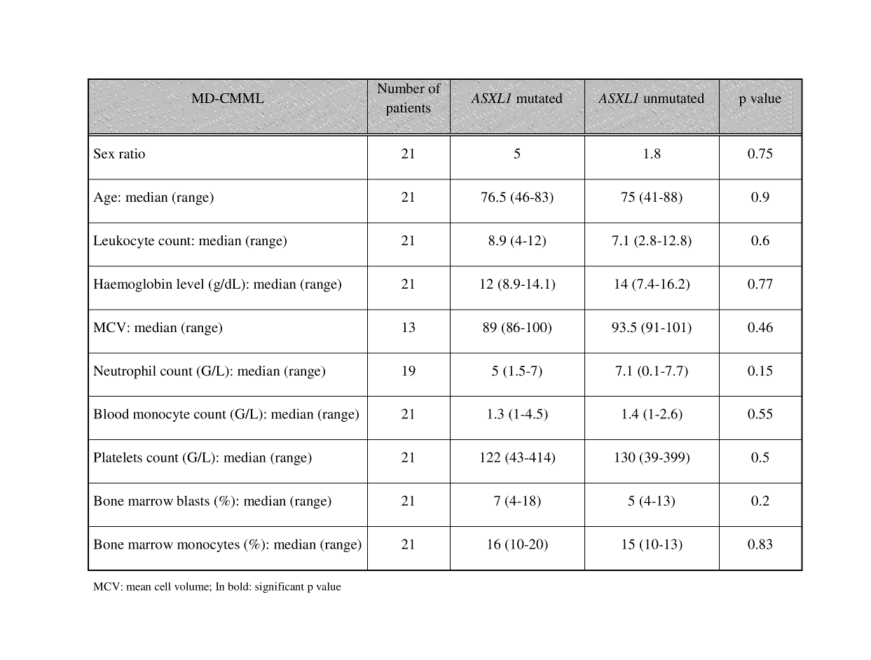

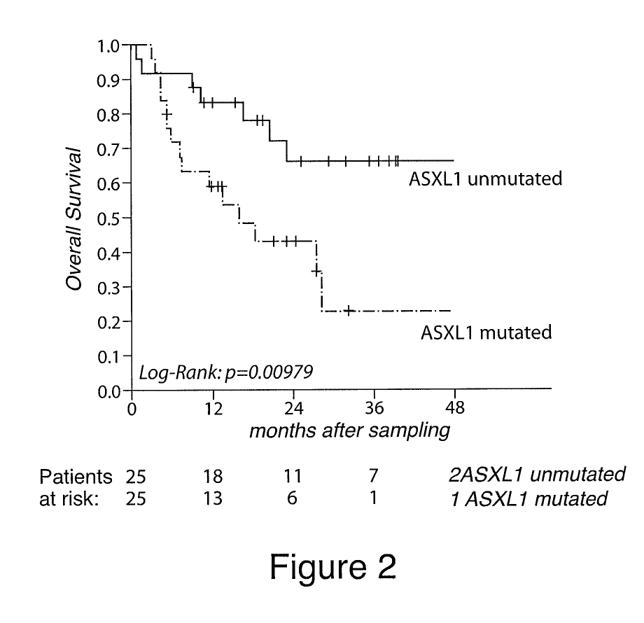

Analysis of overall survival (median follow-up of 29.5 months) was done for the 53 patients and the determined median overall survival was 27.6 months. ASXL1 status could be determined at the time of sampling for 51 patients. Kaplan-Meier analysis showed a lower overall survival rate in the ASXL1 mutated patients (FIG. 2), with no significant impact of acute transformation on overall survival (data not shown). The results have also shown that with respect to MP/MD form, only a trend to a better survival of the MD patients was observed (data not shown).

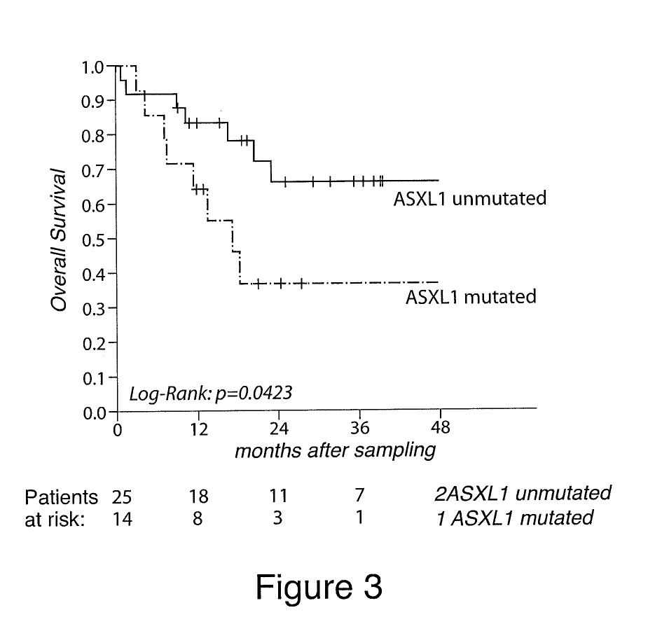

To determine whether ASXL1 had prognostic impact independently of acute transformation, we compared the overall survival of patients mutated for ASXL1 but who had not experienced acute progression to that of unmutated patients: ASXL1 mutation was associated with a poor outcome (FIG. 3). Finally, within MP-CMML patients, cases mutated for ASXL1 had a poorer survival than the unmutated cases. We did the same analysis for TET2 mutational status. In contrast to ASXL1, TET2 had no impact on overall survival (data not shown).

3) Alteration of the ASXL1 Gene in Patients Suffering from MPN

Mutation of ASXL1 in MPN

To determine whether ASXL1 could be involved in other types of myeloid diseases, we studied the ASXL1 gene in 64 myeloproliferative neoplasms (MPNs).

Our series comprised 10 cases of polycythemia vera, 35 cases of essential thrombo-cythemia (ET), 10 cases of primary myelofibrosis (PMF), 1 case of prefibrotic PMF, 5 MPNs at blast phase and 3 unclassifiable MPNs.

We also searched for mutations in 12 non-MPN cases comprising 7 secondary thrombocytosis and 5 secondary erythrocytosis. All patients signed an informed consent and the study was approved by our ethical committee.

We searched for ASXL1 mutations as described previously, and simultaneously for JAK2 (V617F) and TET2 (all exons) mutations.

The results have shown that heterozygous TET2 frameshift mutations are found in 4 out of the 64 MPN cases (6.2%), 2 ET and 2 PMF.

We also found heterozygous frameshift mutations of ASXL1 in 5 cases (7.8%) including 1 ET out of 35, 3 PMF out of 10 (1 was in accelerated phase) and 1 acute myeloid leukemia (AML) post-ET. None of the five ASXL1-mutated cases carried a JAK2 V617F mutation and only one of these five cases (a PMF) was also mutated for TET2. The four other TET2 cases did not have a TET2 mutation but two of them showed an abnormal karyotype.

We analyzed the same sequences in DNA extracted from CD34-purified cells of three patients with ASXL1 and/or TET2 mutation in their blood cell DNA (HD-0496, HD-0536 and HD-0540). The same ASXL1 and TET2 mutations were detected in the corresponding CD34 DNA. This is in agreement with what is known of the physiopathology of MPNs and suggests that ASXL1 mutations occur early during disease evolution.

Finally, ASXL1 mutations were found in nearly 8% of patients suffering from MPNs, and more especially in 33% of patients suffering from PMF.

Mutation of ASXL1innon-CML MPNs

The previous analysis was done for 112 new MPN cases comprising 97 PV, ET and MF, 9 blast-phase PV/ET/MF and 6 unclassified MPN and MPN/MDS forms. We also searched for mutations in 32 non-MPN cases comprising 10 reactive thrombocytosis (RT) and 22 reactive erythrocytosis (RE).

ASXL1mutations were found in 13 cases (11.6%). These mutations were all heterozygous and comprised 10 frameshift (including 7 c.1934dupG p.Gly646TrpfsX12) or nonsense mutations presumed to truncate the protein from its C-terminus that includes the plant homeodomain finger domain (PHD) (Table IV).

Disease-specific mutational frequencies were 8% in PV, 4% in ET, 12% in PMF, 66% in post-PV MF, 25% in post-ET MF, 22% in blast-phase PV/ET/MF and 66% in MPN/MDS. TET2 mutations were present in 11 of 112 patients (10%). Interestingly, none of the 32 reactive cases was mutated for ASXL1.

Because differential diagnosis between ET (MPN) and reactive thrombocytosis may be difficult, the presence of an ASXL1 mutation could help in the diagnosis of MPN to exclude a reactive thrombocytosis.

The presence of an ASXL1 mutation did not influence leukocyte count, hemoglobin or hematocrit levels, but platelet count was lower in ASXL1-mutated cases (416×10⁹ cells/liter, p=0.009) as compared to ASXL1 wt (625×10⁹ cells/liter).

Finally, we observed a high incidence of ASXL1 mutation in MF patients including PMF, post-ET MF and post-PV MF, and a low incidence in ET and PV, implying that ASXL1 may be associated with a more aggressive phenotype. Moreover, the proportion of ASXL1 mutations was high in post-PV MF and post-ET MF (66% and 25%, respectively); suggesting that the ASXL1 status might be used to predict the risk of evolution of PV and ET into MF.

4) Alteration of the ASXL1 Gene in Patients Suffering from AML

To determine whether ASXL1 is involved in AML, we used DNA sequencing and aCGH as described previously to search for mutations and deletions of the gene in 46 cases of AML with normal karyotype and 17 cases with trisomy 8 (n=14), 9q deletion (HD-0632), trisomy 11 (HD-0304) or 20q11-13 deletion (HD-0381) as a sole karyotypic abnormality.

The 63 AMLs were 46 primary cases and 17 transformations of a previous myeloid disease. We did not include therapy-related AMLs. All patients signed an informed consent and the study was approved by our institutional review board.

In all, 41 out of the 50 cases studied by aCGH did not show any copy number aberration (isolated trisomy 8 was not taken into account).

We found heterozygous nonsense or frameshift mutations of ASXL1 in 11 out of the 63 cases (17.5%). We also found several cases of substitution, which we did not take into account because they may represent polymorphisms. The deletion of chromosomal region 20q11-13 (HD-0381), which was visible on the karyotype, involved ASXL1. Thus, in total, 12 cases out of 63 showed ASXL1 alteration (19%).

In two cases, we sequenced ASXL1 in DNA extracted from buccal smears of the patient with ASXL1 mutation; ASXL1 was not mutated, showing that the mutation was acquired.

In agreement with what is known for AML with normal karyotype, 5 almost half of the cases (28 out of 63, 44%) showed exon 12 NPM1 mutations. NPM1 mutations were mutually exclusive with ASXL1 alterations: none of the 28 NPM1-mutated cases showed ASXL1 mutation or deletion, whereas 12 out of the 35 (34%) non-NPM1-mutated cases were mutated or deleted for ASXL1.

FLT3 mutations and internal tandem duplications (ITD) were found in 19 cases (30%) and were not observed with ASXL1 mutations except in one case (HD-0282).

Three cases were mutated in either K or NRAS, and one case in JAK2. Data on CEBPA, available for 11 cases, revealed no mutation.

Actually, the difference between NPM1 and ASXL1 mutations may rather reflect two different routes of leukemogenesis than two alternate hits on the same route. NPM1 mutations are uncommon in AMLs secondary to a chronic myeloid disease. In contrast, 9 of the 17 secondary AMLs (i.e. 53%) showed ASXL1 mutation or deletion (vs 3 of 46 primary AMLs, including a case with mixed lineage dysplasia) and ASXL1 alterations were prominently observed in AMLs secondary to a chronic myeloid disease (9 of 12, 75%), whereas it was not the case for NPM1 mutations (2 of 28).

Were this hypothesis validated by the study of more cases, the detection of ASXL1 mutations would help distinguish between primary and secondary AMLs, and consequently help orient the prognosis, even in the absence of known chronic phases.

In contrast, TET2 mutations in a series of AML were found to correlate neither with the presence or absence of NPM1 or FLT3 mutations nor with an antecedent of chronic myeloid disease. 10 In another series, three of four TET2 mutations were found in secondary cases.

Although TET2 and ASXL1 may function in similar pathways of epigenetic regulation, there might be differences in their relationship with NPM1 and in their window of action during the course of the disease.

In the case that has both ASXL1 and FLT3 alteration, we determined that the ASXL1 mutation, but not the FLT3 ITD, was present at the chronic phase, suggesting that the ASXL1 and FLT3 mutations can cooperate in rare cases.

Finally, the rare RAS mutations occurred indifferently in primary or secondary cases; one was found in an ASXL1—mutated case, which was not unexpected, as we have previously shown that RAS and ASXL1 mutations can co-occur.2 It will now be interesting to determine what other alterations can associate with either the NPM1 or ASXL1 route of leukemogenesis.

5) Frame Shift p.Gly646Trpfsx12 Mutation is Strongly Correlated with RAEB2

All patients signed an informed consent and the study was approved by our institutional review board. They include 65 cases of MDS. According to the WHO criteria, the panel comprised 5 refractory anemia (RA), 13 refractory anemia with ring sideroblasts (RARS) (including one with myelofibrosis), 7 refractory cytopenia with multilineage dysplasia (RCMD), 16 refractory anemia with excess of blasts type 1 (RAEB1), 19 refractory anemia with excess of blasts type 2 (RAEB2) and 5 MDS-unclassified (MDS-U) cases. Six cases were secondary to hematopoietic or non-hematopoietic diseases. The majority of MDS samples were collected at the time of diagnosis; some were in therapeutic abstention of a known MDS and some were under symptomatic treatment. Seventeen cases were IPPS low risk (0), 23 were int-1 (0.5-1), 12 were int-2 (1.5-2) and 7 were high risk (≧2.5).

DNA sequencing of exon-coding sequences of ASXL1, CBL, FLT3, IDH1, IDH2, JAK2, KRAS, NPM1, NRAS, RUNX1, TET2 and WT1 was done as follows. PCR amplifications of bone marrow cell DNA were done in a total volume of 25 μl PCR mix containing at least 5 ng template DNA, Taq buffer, 200 μmol of each deoxynucleotide triphosphate, 20 pmol of each primer and 1 unit of Hot Star Taq (Qiagen). PCR amplification conditions were as follows: 95° C. 10 min; 95° C. 30 sec, 55° C. 30 sec, 72° C. 30 sec to 1 min depending on PCR product length for 35 cycles; 72° C. 10 min. PCR products were purified using MILLIPORE plate MSNUO30. One microliter of the purified PCR products was used for sequencing using the Big Dye terminator v1.1 kit (APPLIED BIOSYSTEMS) including the forward or reverse primer. After G50 purification, sequences were loaded on an ABI 3130XL automat (APPLIED BIOSYSTEMS). The sequence data files were analyzed using both SEQSCAPE and PHRED/PHRAP/CONSED softwares and all mutations were confirmed on an independent PCR product.

ASXL1 exon 12 frameshift mutations (11 times the same p.Gly646Trpfsx12) were observed in 12 out of the 65 MDS cases (18.5%) including 1 out of 5 RA (20%), 2 out of 16 RAEB1 (12.5%) and 9 out of 19 RAEB2 (47.4%).

We found 12 cases with TET2 mutation (18.5%) and 4 with RUNX1 mutation (6.2%). One patient (HD-0311) had two TET2 mutations. TET2 mutations were frequent in RAEB1 (7/16, 43.8%). Mutations in RUNX1 and TET2 were mutually exclusive but both could associate with ASXL1 mutations: two cases showed both an ASXL1 and a TET2 mutation and three cases both an ASXL1 and a RUNX1 mutation. One case of ASXL1 deletion (HD-0190) and one case of TET2 deletion (HD-0145) have been reported. One case (HD-0232) had a break in RUNX1 detected by aCGH (not shown).

We did not find any FLT3, NPM1 or WT1 mutation. One MDS-U had a JAK2 mutation and one RCMD case a KRAS mutation. Five cases, all RAEB2, were mutated in CBL. In one of these the mutation was homozygous. One substitution occurred in the case with trisomy 11 (HD-0264), and showed a 2/3 ratio with the wild-type residue, suggesting that the mutated allele was duplicated.

We found 5 IDH mutations in the 65 cases (7.7%), including 2 mutations in IDH1 and 3 in IDH2.

Based on the known functions of the proteins, on a previous model and classification on where the mutations were present (MDSs and/or secondary AMLs and/or primary AMLs) and on how they combined, we tentatively grouped the genes in four classes.

The first class (we called “initiators”) includes RUNX1 and TET2. They may cause clonal dominance of hematopoietic stem cells.

ASXL1 and NPM1 would constitute class II (“selectors”). Mutations in these genes may select a leukemogenic pathway leading towards either primary or secondary AML.

Genes associated with proliferation (CBL, FLT3, JAK2, RAS) define class III (“amplifiers”). JAK2 mutation plays little role in MDSs and NK-AMLs.

Finally, for three reasons we grouped IDH1, IDH2, and WT1 in a putative class IV we provisionally called “boosters”. First, IDH and WT1 mutations were exclusive but could co-occur with mutations in genes from other classes. Second, they occurred primarily in AMLs and were rare in MDSs (and also in myeloproliferative neoplasms). Third, mutations of these genes could be associated with modifications of the HIF1 and oxygen-sensing pathways. Class IV mutations are rather associated with acute phase.

Overall, AML cases had zero, one or three mutations. Because of this, and although no case had four mutations, we propose that AML develops following—at least—four cooperating mutations, one from each class. This is speculative and the identification of new target genes and the study of others will lead to a more precise picture.

1. A method for diagnosing a myeloid cancer in a subject, which comprises the step of analyzing a biological sample from said subject by determining the presence or the absence of a mutation in the ASXL1 (additional sex combs like 1) gene coding for the polypeptide having the sequence SEQ ID No 2, wherein the presence of such a mutation is correlated with a myeloid cancer.

2. The method of claim 1, wherein said subject is a human.

3. The method of claim 1, wherein said myeloid cancer is selected from myelodysplastic syndrome (MDS), myelodysplatic/myeloproliferative disorders, myeloproliferative neoplasm (MPN) and acute myeloid leukemia (AML).

4. The method of claim 3, wherein said myeloid cancer is myelodysplastic syndrome (MDS), which is a refractory anemia with excess of blasts type 2 (RAEB2).

5. (canceled)

6. The method of claim 3, wherein said myeloid cancer is a myelodysplatic/myeloproliferative disorder, which is a chronic myelomonocytic leukemia (CMML), or a myeloproliferative neoplasm (MPN), which is selected from the group consisting of a primary myelofibrosis (PMF), a post-polycythemia vera myelofibrosis (post-PV MF), and a post-essential thrombocythemia myelofibrosis (post-ET MF).

7. (canceled)

8. The method of claim 6, wherein said CMML is a myeloproliferative form of CMML (MP CMML).

9.-13. (canceled)

14. The method of claim 3, wherein said myeloid cancer is MPN, which is essential thrombocythemia (ET), and the presence of a mutation in the ASXL1 gene exclude a reactive thrombocytosis.

15. The method of claim 3, wherein said myeloid cancer is an acute myeloid leukemia (AML) in a subject, which is a secondary AML.

16. (canceled)

17. The method of claim 15, wherein said secondary AML follows a chronic myeloid disease.

18.-20. (canceled)

21. The method of claim 1, wherein said mutation results in the expression of a mutated ASXL1 protein, said mutated ASXL1 protein does not comprise any PHD domain (SEQ ID No 3) or a fragment thereof.

22. The method of claim 21, wherein said mutation is a non sense mutation leading to the introduction of a stop codon (X) in the open reading frame of the ASXL1 protein.

23. The method of claim 22, wherein said mutation is a non sense mutation selected in the group comprising Tyr591X, Gln592X, Lys618X, Arg693X, Gln759X, Gln768X, Leu775X and Arg1068X.

24. (canceled)

25. The method of claim 22, wherein said mutation is an insertion or deletion inducing the expression of the mutated ASXL1 protein having a mutation selected in the group comprising Gly64Trp FS, Arg596Pro FS, Ala611Arg FS, His630Pro FS, Gly646Trp FS, Leu762Phe FS, Trp796Gly FS, Thr822Asn FS, Thr836Leu FS, Ser846Gln FS, Asp879Glu FS, Lys888Glu FS, Leu1213Ile FS, Pro1263Gln FS, Leu1266H is FS, Trp1271Lys FS, or Ser1457Pro FS.

26.-28. (canceled)

29. A kit for diagnosing myeloid cancer in a subject comprising at least one nucleic acid probe or oligonucleotide or at least one antibody, which can be used in a method as defined in claim 1 for determining the presence or the absence of a mutation in the ASXL1 (additional sex combs like 1) gene coding for the polypeptide having the sequence SEQ ID No 2, wherein the presence of such a mutation is correlated with a myeloid cancer.

30.-31. (canceled)

32. A method for the estimating the prognosis of a myeloid cancer, in a subject, which comprises the step of analyzing a biological sample from said subject by determining the presence or the absence of a mutation in the ASXL1 (additional sex combs like 1) gene coding for the polypeptide having the sequence SEQ ID No 2, wherein the presence of the mutation is indicative of a poor prognosis of said patient, and the absence of the mutation is suggestive of a good prognosis of said patient.

33. The method of claim 32, wherein said myeloid cancer is a myelodysplastic syndrome (MDS), which is a refractory anemia with excess of blasts type 2 (RAEB2).

34.-35. (canceled)

36. The method of claim 32, wherein said myeloid cancer is a MDS and the method is for the prognosis of the progression of said MDS to AML, the presence of an ASXL 1 mutation being indicative of such a risk progression.

37. (canceled)

38. The method of claim 32, wherein said myeloid cancer is a CMML and the method is for the prognosis of the progression of said CMML to AML, the presence of an ASXL 1 mutation being indicative of such a risk progression.

39.-44. (canceled)

45. A method for predicting the response to a treatment for a myeloid cancer in a subject, which comprises the step of analyzing a biological sample from said subject by determining the presence or the absence of a mutation in the ASXL1 (additional sex combs like 1) gene coding for the polypeptide having the sequence SEQ ID No 2.

46.-47. (canceled)
